(19) United States
(12) Patent Application Publication
(10) Pub. No.: US 2005/0214242 A1
(43) Pub. Date: Sep. 29, 2005

(54) Compositions containing internally activated antioxidant

(76) Inventors: Fatemeh Mohammadi, Hauppauge, NY (US); Anna Czarnota, Huntington, NY (US); Ismail A. Syed, Ronkonkoma, NY (US)

Correspondence Address: THE ESTEE LAUDER COS, INC, 125 PINELAWN ROAD, MELVILLE, NY, 11747, US

(21) Appl. No.: 11/043,916

(22) Filed: Jan. 25, 2005

Related U.S. Application Data
(60) Provisional application No. 60/540,561, filed on Jan. 30, 2004.

(51) Int. Cl. A61K 7/06

(52) U.S. Cl. 424/74

(57) ABSTRACT

The present invention provides a method of activating a heat-activated component by an internal source of heat. As a result of an exothermic reaction between a combination of a heat-generating component and the heat-activated component, a source of heat is created within the composition without the use of an external heating source such as a hair dryer or exposure to the sun. The heat-generating component provides a heat release that triggers the activity of the heat-activated component. The compositions of the present invention are self-contained systems for generating heat to trigger the heat-activated component, and therefore, do not rely upon an external source of heat to affect the heat-activated component.

This application claims priority from U.S. Pat. No. 60/540561, filed Jan. 30, 2004.

### FIELD OF THE INVENTION

The present invention relates to cosmetic compositions containing a heat-activated component triggered by a heat source internally present in the composition. More specifically, the invention relates to compositions containing a heat-generating agent to trigger the activation of an encapsulated heat-activated antioxidant for treating the skin surface after topical application.

### BACKGROUND OF THE INVENTION

Heat-activated ingredients are best known in hair care products, pigmented compositions, adhesives and more recently gene therapy. In the case of hair care products, for example, an external heat source such as a hair dryer or exposure to the sun activates the heat-activated ingredients to provide hair care benefits. Heat-activated ingredients in hair care compositions are beneficial because they transform the normally damaging effects of heat on the hair into part of a positive process of providing treatment benefits to the hair. When the application of heat is used to activate a heat-activated ingredient, hair is substantially less or not damaged by the heat. Another type of heat-activated ingredient is a thermochromic pigment which can be used as an indicator, for example, by changing color to indicate that a surface or object is becoming too hot. Whether it's a pigment, an adhesive, or a hair care product, the one thing these products have in common is that the heat-activated component relies on the application of an external source of heat to activate it.

Like heat-activated ingredients, the addition of heat-generating agents to compositions is known, but is generally for creating a warm sensation on the skin. When a heat-generating agent undergoes a reaction in a composition it provides an instantaneous rise in temperature, for example, 1 to 10° C. or more. The exothermic reaction is between the heat-generating active in the composition and an external heat-generating trigger, for example, either water or peroxide. This is similar to the application of an external heat source to activate the heat-activated ingredient because the catalyst for the generation of heat is external and it is a one step process.

It has not heretofore been known to combine the heat-generating agent with a heat-activated ingredient such that they are internal, and therefore, are components that interact with one another within a composition. Typically, regardless of the heat-activated ingredient employed, heat is provided as an external source to the composition. In the case of compositions containing heat-generating agents, the heat creates a warm sensation on the skin and is generated by an external source of water or peroxide. It would be beneficial to have these components work together in a chain reaction within the composition and provide additional treatment benefits to the skin surface. Therefore, there is a need for a product that generates heat internally within the composition as a trigger for thermosensitive agents also contained therein and to provide treatment for the surface of the skin. The present invention introduces the concept of self contained system such that a heat-activated ingredient is internally activated to provide beneficial activity to the surface of the skin.

### SUMMARY OF THE INVENTION

The invention relates to a composition comprising an effective amount of a heat generating component of at least one heat-generating agent and a heat-activated component containing at least one encapsulated heat-activated ingredient. The heat-activated component is stimulated by heat emanating from the heat-generating agent. The two reactive components render a treatment effect on the surface of the skin solely by an internal reaction, and therefore, is a self contained system. The heat-activated ingredient agent is encapsulated yet can be triggered by the heat-generating agent. The heat-generating agent undergoes an exothermic reaction when combined with an external reactant and becomes in turn a reactant for the heat-activated ingredient agent. The heat generating component may contain one or more heat generating agents especially those that are water reserving or sorbing based agents (i.e., agents that are capable of sorbing water exothermically) for extending the duration of time that heat is released. The heat generating agent can be encapsulated or unencapsulated.

In addition, the present invention includes methods of topically applying the components of the composition to the skin to produce the exothermic reaction, and thus, trigger the thermoreactive skin treatment activity on the skin surface. The methods of the present invention include softening the skin, oxygenating the skin and protecting the skin from damage to the barrier function caused by the presence of free radicals.

### DETAILED DESCRIPTION OF THE INVENTION

The present invention, in its various embodiments, is predicated upon the surprising discovery that effective amounts of a heat generating component comprising one or more heat generating agents undergoes an exothermic reaction producing an internal heat source that triggers an encapsulated heat-activated ingredient, preferably skin treatment active. The compositions of the present invention contain a heat-activated ingredient that can be for example, any type of ingredient that exhibits a change in the rate of its activity due to the presence of heat. The heat-activated ingredient becomes active or increases its level of activity when it is heated to a temperature range of between 40 and 60° C. Essentially, the heat-activated ingredient is thermosensitive because it only becomes active when its activity is triggered by heat energy as indicated by a rise in temperature (measure of the concentration of heat). Any type of heat activated or heat stable component can be used in the present invention. For example, heat activated components can be lipophilic or hydrophilic in nature. Examples of activities contemplated within the scope of the present invention that may be associated with the heat-activated component include, but are not limited to, antioxidant, anti-acne, antimicrobial, skin whitening or bleaching, skin conditioning or moisturization, skin protectant, sunscreen, UV absorber, anti-cellulite, anti-fungal, anti-inflammatory, anti-wrinkle, artificial tanning, barrier repair, skin brightening, skin feel modification, exfoliation, soothing and/or healing, topical anesthetic, and other biological actives for treatment of the skin. Preferably, the heat-activated ingredient is not related to color change as an activity.

A preferred heat-activated ingredient agent is a ferment of Thermus thermophilus strain Gy1211 (Gy1211) from a deep-sea hydrothermal vent in Guaymas Basin (Gulf of California at 2000 m depth). Eight new deep-sea isolates have been isolated from the sample source, however, Gy1211, for purposes of characterization has been used as the representative strain. Many strains of Thermus are isolated inland, from continental hot springs. These sources are neutral or alkaline and have very low salinity. It has recently been found that Thermus isolated from shallow water marine hot springs in Iceland which normally occur on the seashore or at shallow depths where the vent water is mixed with seawater, are more halotolerant than Thermus originating from terrestrial hot springs. It is noted that the deep-sea isolates form pleomorphic rods, about 0.5 to 1.0 micron in diameter and about 5 to 10 micron in length. Spores are not observed and long filaments are seen in liquid cultures. Further, both vesicular and aggregate types of rotund bodies can be observed. Characterization of Gy1211 is presented in Marteinsson et al. “Isolation and characterization of Thermus thermophilus Gy1211 from a deep-sea hydrothermal vent”, Extremophiles (1999) 3:247-251.

The fermentation media for Gy1211 is disclosed in WO 02/066668 A2 for producing proteins and use of fermentation media for Gy1211 in cosmetic compositions to modulate the cutaneous concentration of ceramides, to stimulate the immune system to provide protection as a detoxifying agent and against free radicals, especially oxygen peroxide. The reference describes the fermentation media for Gy1211 as having catalase-like activity, however, there is no mention in the reference of triggering an enhanced antioxidant effect by the presence of a heat source internally placed in the composition. Nor is there any mention that the activity of the fermentation media for Gy1211 can be activated when the Gy1211 ferment is encapsulated.

The Gy1211 ferment used in the present invention is prepared according to microbiological techniques disclosed in WO 02/066668, the contents of which are incorporated herein by reference. In summary, the bacteria can be cultured on medium 162 containing 2% (w/v) agar and 1% (w/v) NaCl (designated 162-1). The Gy1211 ferment is grown at a temperature of between about 72 to 75° C., under agitation, with a pH of about 7.2. Microbiological procedures are described in WO 02/066668 as having been optimized. The Gy1211 ferment is available commercially from Sederma, Le Perray-en Yvelines Cedex, France under the name Venuceane™ and is provided in the form of a liquid, but can also be a powder if freeze dried or a gel. The Gy1211 ferment can be added directly to a composition or it can be encapsulated. The amount of the heat-activated ingredient is about 0.5 to 5 percent, preferably about 1 to 4 percent.

In a preferred embodiment of the present invention, the heat-activated ingredient is encapsulated. Preferably, when the heat-activated ingredient is Venuceane™ it is encapsulated in nylon 6/12 available, for example, under the commercial name Orgasol® 4000 Exd Nat Cos. in nearly a 1:1 ratio. The encapsulated venuceane is available from Lipo Chemicals, Inc., Paterson, N.J. The Orgasol® 4000 Exd Nat Cos is known to have good absorption of excess sebum and therefore when used in the present invention to encapsulate venuceane, the resulting product is beneficial as an oil/sebum control product. Surprisingly, even when the heat-activated ingredient is encapsulated, the heat-generating agent of the present invention is capable of triggering the activity of the heat-activated ingredient. The typical skin surface that is not subjected to excessive cold or hot temperature is about 30 to 35° C. Therefore, the source of heat for the heat-activated component of the present invention is not maximally provided by the keratinous surface to which it is topically applied. The heat-generating agent is internal to the composition (i.e., self-contained) and produces an increase in temperature of about at least about 15° C., preferably about 20° C., and most preferably about 25° C.

Any kind of a heat-generating agent can be used in the present invention. For example, catalase (and peroxide), chlorides such as CaCl2, MgCl2, AlCl3, FeCl3, ZnCl2; sulfates such as those including magnesium, zinc, calcium, iron, aluminum, and sodium; dry aluminum, oxides such as those including calcium and magnesium, carbonates, sodium hydrogenphosphate and the like. The heat-generating agent can also include, for example, kaolin, urea, silica gel, iron powder, activated alumina, solid adsorbent materials capable of sorbing water exothermically that are known in the art, e.g., untreated, treated, or synthetic zeolite (alkali metal aluminosilicates, e.g., sodium silicoaluminate), and combinations thereof. The heat generating agent may require the presence of a catalyst such as, for example, water or in the case of catalase, a peroxide. Preferably, the heat generating component comprises at least one calcium chloride and the catalyst is external water.

In a preferred embodiment of the present invention, the heat-generating agent is encapsulated. The heat-generating agent can be encapsulated by surface treating calcium chloride with a combination of dimethicone and silica. An example of the encapsulated heat-generating agent is Natural Hot™ TR available from Resources of Nature, Inc., South Plainfield, N.J. Encapsulation permits the release of heat to be gradual and gentle on the skin surface rather than a blast of heat at the point of application. The term “heat-generating effective amount” as used herein refers to an amount of encapsulated heat generating agent in the heat generating component that brings about a release of heat sufficient to trigger the heat-activated skin treatment active. Accordingly, the amount of encapsulated heat generating agent suitable in the compositions of the present invention is about 0.2 to about 40.0 percent, and preferably about 0.5 to about 25.0 percent by weight of the composition. The unencapsulated heat generating agent is present in an amount of about 0.1 to about 4.0 percent, and preferably about 0.2 to about 2.0 percent by weight of the composition.

The heat activated effect of the present invention involving the heat-generating agent can be achieved in any type of cosmetically or pharmaceutically acceptable vehicle for topical application with which the encapsulated heat-activated component and the heat-generating component are compatible, e.g., a gel, a cream, a lotion, facial and body masks, an ointment, a mousse, a spray, a solid stick, a powder, a suspension, a dispersion, and the like. Preferably, however, the products are a massage cream or lotion for the body, especially as a spa products such as hand or foot massage products, body oils and products; shave cream and products; cleansers; masks; and hair products such as scalp treatments, shampoos and conditioners. The compositions of the invention are applied to the skin in a manner appropriate to the intended desired end result in terms of heat generation to trigger the heat-activated ingredient agent. About 1 to 2 times per week the compositions of the present invention are applied to the skin. Before application, however the skin is dampened. After application, the compositions are massaged gently into the skin during which time the heat generation and self-contained activation occurs. Upon completion of the massage and the heat activation, the composition is rinsed off of the skin.

The invention is further illustrated by the following non-limiting examples:

### EXAMPLE I

The following is a composition according to the present invention:

| Ingredients | |
|---|---|
| Isonoyl isononanoate | 17.25 |
| Benzotrizoyl dodecyl p-cresol | 0.05 |
| Preservative | 0.40 |
| Isododecane | 5.00 |
| Polyethylene | 5.00 |
| Emulsifying elastomer | 40.00 |
| Pigment | 1.00 |
| Silica | 0.30 |
| Polysorbate 21 | 3.00 |
| Polysorbate 60 | 3.00 |
| Encapsulated venuceane | 5.00 |
| Encapsulated heating agent | 20.00 |

Combine the ingredients except for the encapsulated venuceane and the encapsulated heating agent in the order provided and mix. Add the encapsulated venuceane to the mixture and mix. Finally, add the encapsulated heating agent to the mixture and mix.

### EXAMPLE II

| Ingredients | |
|---|---|
| Isononoyl isononanoate | 11.75 |
| Benzotrizoyl dodecyl p-cresol | 0.05 |
| Preservative | 0.30 |
| Isododecane | 7.50 |
| Emulsifying elastomer | 50.00 |
| Pigment | 0.30 |
| Silica | 0.10 |
| Polysorbate 21 | 2.50 |
| Polysorbate 60 | 2.50 |
| Encapsulated Venuceane ™ | 5.00 |
| Encapsulated heating agent | 20.00 |

1. A composition for topical application to the skin comprising at least one heat-activated component internally stimulated by a heat-generating component within the composition.

2. The composition of claim 1 in which said heat-activated component is derived from a non-colorant active ingredient.

3. The composition of claim 2 wherein said active is a Thermus thermophilus ferment.

4. The composition of claim 1 wherein said heat-activated component is encapsulated.

5. The composition of claim 1 wherein said heat-activated component activity is triggered by an increase in temperature of at least about 2° C. per minute in a period of about 5 minutes.

6. The composition of claim 1 wherein said heat-generating agent is derived from an exothermic reaction using at least one reactant selected from the group consisting of inorganic compounds, organic compounds or microorganisms.

7. The composition of claim 1 in which said heat-activated component is present in an amount of from about 0.5 to about 5.0 percent by weight of the composition.

8. The composition of claim 6 in which said heat-activated component is present in an amount of from about 1.0 to about 4.0 percent by weight of the composition.

9. The composition of claim 6 wherein said heat-generating agent is selected from the group consisting of calcium chloride, catalase, zeolite, kaolin, magnesium sulfate, urea, silica gel, iron powder, and activated alumina.

10. The composition of claim 9 in which said heat-generating agent is encapsulated.

11. The composition of claim 10 in which said heat-generating agent is an encapsulated calcium chloride.

12. The composition of claim 11 in which said encapsulated calcium chloride is present in an amount of about 1.0 to about 40.0 percent.

13. A method of activating an encapsulated heat-activated agent within a composition which comprises combining a heat-generating agent with at least one encapsulated heat-activated agent, producing an internal heat source with the heat generating agent, and triggering the activity of the heat-activated agent with the internal heat source.

14. A method of treating the skin comprising the step of topically applying the composition of claim 1.
